(19) United States
(12) Patent Application Publication
(10) Pub. No.: US 2026/0191673 A1
(43) Pub. Date: Jul. 9, 2026

(54) BRACE DEVICES, SYSTEMS, AND METHODS

(71) Applicant: Shock Doctor, Inc., Fountain Valley, CA (US)

(72) Inventor: Jay Turkbas, Hideout, UT (US)

(21) Appl. No.: 19/443,227

(22) Filed: Jan. 8, 2026

Related U.S. Application Data
(60) Provisional application No. 63/743,066, filed on Jan. 8, 2025.

(51) Int. Cl. A61F 5/01

(52) U.S. Cl. CPC A61F 5/0125; A61F 2005/0179

(57) ABSTRACT

A brace system is configured to be worn on a limb of a wearer. The brace system includes a support member configured to receive the limb of the wearer and a hinge assembly coupled to the support member. The hinge assembly includes a spring hinge member and a tension member. The spring hinge member and the tension member apply forces to the support member that urge the limb of the wearer in an extension direction.

### CROSS REFERENCE TO RELATED APPLICATION

The present application claims the benefit of and priority to, under 35 U.S.C. § 119(e), U.S. Provisional Application Ser. No. 63/743,066, filed on Jan. 8, 2025, which is hereby incorporated herein by reference in its entirety for all that it teaches and for all purposes.

### TECHNICAL FIELD

The present disclosure relates to devices and methods for supporting a limb or limbs of a user when worn. More specifically, the present disclosure relates to devices, systems, and methods for supporting a knee of a user when worn.

### BACKGROUND

Devices for supporting or stabilizing the limb or limbs of a wearer may be worn by a user for everyday use and/or for use when engaging in physical activity. Injuries to a limb or limbs are common and may affect a user's physical ability and/or athletic performance. For certain users it may be beneficial to use an artificial structure to support a limb that has been weakened or injured. Certain rigid or flexible structures may be worn through the day and/or when engaging in physical activity to provide structural support, or prevent injury. In certain instances they may assist a wearer's movement, reduce weight bearing forces, or provide easier movement capability to a wearer.

For example, an athletic sleeve may be worn around a wearer's limb to provide compression to the limb. An athletic sleeve may also be worn around a wearer's joint such as a knee, wrist, ankle, or elbow to provide lateral or torsional support to the joint and/or to hold the joint in alignment. In some cases, an athletic sleeve may hold snugly to a joint such as a knee or elbow and improve performance while the wearer is engaged in physical activity. However, in some cases, an athletic sleeve may not provide adequate support to a wearer who has suffered an injury or may not provide targeted support to a wearer whose joint may require support in a specific location.

Certain support structures for the leg or knee of a user, such as sleeves, straps, or braces, are available and may provide certain advantages, such as weight bearing capabilities. However, such weight bearing capabilities are typically unchangeable for individual support structures, rendering them less effective for wearers with different training requirements, rehabilitation requirements, and/or preferences.

### SUMMARY

In one example (“Example 1”), a brace system is configured to be worn on a limb of a wearer. The brace system includes: a support member configured to receive the limb of the wearer; a hinge assembly coupled to the support member, the hinge assembly including: a spring hinge member; and a tension member; wherein the spring hinge member and the tension member apply forces to the support member that urge the limb of the wearer in an extension direction.

In another example (“Example 2”), the brace system of Example 1, wherein the spring hinge member includes a serpentine spring element.

In another example (“Example 3”), the brace system of Example 2, wherein the spring hinge member further includes an upper base coupled to the support member and a lower base coupled to the support member, the serpentine spring element coupling the upper base to the lower base

In a further example (“Example 4”), the brace system of Example 2, wherein the tension member is positioned on an anterior side of the serpentine spring element.

In another example (“Example 5”), the brace system of Example 4, wherein the spring hinge member further includes a plurality of grooves on the anterior side of the serpentine spring element, the plurality of grooves receiving and positioning the tension member along the serpentine spring element

In a further example (“Example 6”), the brace system of Example 2, wherein the spring hinge member further includes a plurality of partial chocks coupled to the serpentine spring element, the plurality of partial chocks receiving and positioning the tension member along the serpentine spring element.

In another example (“Example 7”), the brace system of Example 6, wherein the tension member is positioned between adjacent partial chocks.

In a further example (“Example 8”), the brace system of Example 6, wherein the plurality of partial chocks includes first partial chocks and second partial chocks, each of the first partial chocks including a first concave surface contacting the tension member and facing in a first direction, and each of the second partial chocks including a second concave surface contacting the tension member and facing in a second direction opposite the first direction.

In another example (“Example 9”), the brace system of Example 1, wherein the spring hinge member includes a plurality of partial coils, wherein adjacent partial coils extend in opposite directions.

In a further example (“Example 10”), the brace system of Example 1, wherein the spring hinge member further includes an upper bollard receiving an upper end portion of the tension member and a lower bollard receiving a lower end portion of the tension member.

In another example (“Example 11”), the brace system of Example 10, wherein the upper end portion of the tension member includes an upper loop received by the upper bollard and the lower end portion of the tension member includes a lower loop received by the lower bollard.

In a further example (“Example 12”), the brace system of Example 1, wherein the tension member is selectively attachable to and detachable from the spring hinge member.

In another example (“Example 13”), the brace system of Example 1, wherein the tension member is a first tension member that is selectively attachable to the spring hinge member and the first tension member has a first deformation resistance characteristic when attached to the spring hinge member, and further including a second tension member that is selectively attachable to the spring hinge member and the second tension member has a second deformation resistance characteristic when attached to the spring hinge member, the second deformation resistance characteristic being different than the first deformation resistance characteristic.

In a further example (“Example 14”), the brace system of Example 1, wherein the brace system is a knee brace system, and the limb of the wearer is a leg of the wearer.

In one example (“Example 15”), a brace system is configured to be worn on a limb of a wearer. The brace system includes: a support member configured to receive the limb of the wearer; a hinge assembly coupled to the support member, the hinge assembly including: a spring hinge member configured to carry a bending load upon flexion of the limb of the wearer; and a tension member configured to carry a tensile load upon flexion of the limb of the wearer.

In another example (“Example 16”), the brace system of Example 15, wherein the spring hinge member includes a serpentine spring element.

In a further example (“Example 17”), the brace system of Example 16, wherein the spring hinge member further includes an upper base coupled to the support member and a lower base coupled to the support member, the serpentine spring element coupling the upper base and the lower base.

In another example (“Example 18”), the brace system of Example 16, wherein the tension member is positioned on an anterior side of the serpentine spring element.

In a further example (“Example 19”), the brace system of Example 18, wherein the spring hinge member further includes a plurality of grooves on the anterior side of the serpentine spring element, the plurality of grooves receiving and positioning the tension member along the serpentine spring element.

In another example (“Example 20”), the brace system of Example 16, wherein the spring hinge member further includes a plurality of partial chocks coupled to the serpentine spring element, the plurality of partial chocks receiving and positioning the tension member along the serpentine spring element.

In a further example (“Example 21”), the brace system of Example 20, wherein the tension member is positioned between adjacent partial chocks.

In another example (“Example 22”), the brace system of Example 20, wherein the plurality of partial chocks includes first partial chocks and second partial chocks, each of the first partial chocks including a first concave surface contacting the tension member and facing in a first direction, and each of the second partial chocks including a second concave surface contacting the tension member and facing in a second direction opposite the first direction.

In a further example (“Example 23”), the brace system of Example 15, wherein the spring hinge member includes a plurality of partial coils, wherein adjacent partial coils extend in opposite directions.

In another example (“Example 24”), the brace system of Example 15, wherein the spring hinge member further includes an upper bollard receiving an upper end portion of the tension member and a lower bollard receiving a lower end portion of the tension member.

In a further example (“Example 25”), the brace system of Example 24, wherein the upper end portion of the tension member includes an upper loop received by the upper bollard and the lower end portion of the tension member includes a lower loop received by the lower bollard.

In a further example (“Example 26”), the brace system of Example 15, wherein the tension member is selectively attachable to and detachable from the spring hinge member.

In another example (“Example 27”), the brace system of Example 15, wherein the tension member is a first tension member that is selectively attachable to the spring hinge member and the first tension member has a first deformation resistance characteristic when attached to the spring hinge member, and further including a second tension member that is selectively attachable to the spring hinge member and the second tension member has a second deformation resistance characteristic when attached to the spring hinge member, the second deformation resistance characteristic being different than the first deformation resistance characteristic.

In a further example (“Example 28”), the brace system of Example 15, wherein the brace system is a knee brace system, and the limb of the wearer is a leg of the wearer.

In one example (“Example 29”), a method for reconfiguring a brace system is provided. The brace system includes a support member, a spring hinge member coupled to the support member, a first tension member having a first deformation resistance characteristic when attached to the spring hinge member, and a second tension member having a second deformation resistance characteristic when attached to the spring hinge member, the second deformation resistance characteristic being different than the first deformation resistance characteristic. The method includes: detaching the first tension member from the spring hinge member; and thereafter attaching the second tension member to the spring hinge member.

In another example (“Example 30”), the method of Example 29, wherein the spring hinge member includes a serpentine spring element, and attaching the second tension member to the spring hinge member includes positioning the second tension member along the serpentine spring element.

In a further example (“Example 31”), the method of Example 30, wherein the serpentine spring element includes a plurality of grooves, and attaching the second tension member to the spring hinge member includes positioning the second tension member in the plurality of grooves.

In another example (“Example 32”), the method of Example 30, wherein the spring hinge member includes a plurality of partial chocks coupled to the serpentine spring element, and attaching the second tension member to the spring hinge member includes positioning the second tension member between the plurality of partial chocks.

In a further example (“Example 33”), the method of Example 30, wherein the spring hinge member includes a plurality of bollards coupled to the serpentine spring element, and attaching the second tension member to the spring hinge member includes coupling end portions of the second tension member to the plurality of bollards.

In another example (“Example 34”), the method of Example 30, wherein attaching the second tension member to the spring hinge member includes positioning the second tension member on an anterior side of the serpentine spring element.

In a further example (“Example 35”), the method of Example 29, further including, after attaching the second tension member to the spring hinge member, positioning the brace system on a limb of a wearer.

In another example (“Example 36”), the method of Example 35, wherein the brace system is a knee brace system, and the limb of the wearer is a leg of the wearer.

In one example (“Example 37”), a brace system is configured to be worn on a limb of a wearer. The brace system includes: a support member configured to receive the limb of the wearer; a spring hinge member coupled to the support member and having a medial side, a lateral side, and a width direction extending therebetween, the spring hinge member including: an upper base coupled to the support member; a lower base coupled to the support member; and a serpentine spring element coupling the upper base to the lower base, the serpentine spring element urging the limb of the wearer in an extension direction, and the serpentine spring element having a varying width.

In another example (“Example 38”), the brace system of Example 37, wherein the serpentine spring element includes: an upper partial coil coupled to the upper base; a lower partial coil coupled to the lower base; and an intermediate partial coil disposed between the upper partial coil and the lower partial coil, the intermediate partial coil having a maximum width of the serpentine spring element.

In a further example (“Example 39”), the brace system of Example 37, wherein the medial side of the serpentine spring element follows a curved path when viewed from an anterior side of the spring hinge member.

In another example (“Example 40”), the brace system of Example 39, wherein the lateral side of the serpentine spring element follows a curved path when viewed from the anterior side of the spring hinge member.

In a further example (“Example 41”), the brace system of Example 37, wherein the lateral side of the serpentine spring element follows a curved path when viewed from an anterior side of the spring hinge member.

In another example (“Example 42”), the brace system of Example 37, further including a tension member positioned on an anterior side of the serpentine spring element.

In a further example (“Example 43”), the brace system of Example 37, wherein the brace system is a knee brace system, and the limb of the wearer is a leg of the wearer.

In one example (“Example 44”), a brace system is configured to be worn on a limb of a wearer. The system includes: a support member configured to receive the limb of the wearer; a spring hinge member coupled to the support member and having a medial side, a lateral side, and a width direction extending therebetween, the spring hinge member including: an upper base coupled to the support member; a lower base coupled to the support member; a plurality of partial coils coupling the upper base and the lower base, the plurality of partial coils including: an upper partial coil having an upper width; a lower partial coil; and an intermediate partial coil disposed between the upper partial coil and the lower partial coil, the intermediate partial coil having an intermediate width, and the intermediate width being greater than the upper width.

In another example (“Example 45”), the brace system of Example 44, wherein the lower partial coil has a lower width, the lower width being less than the intermediate width.

In a further example (“Example 46”), the brace system of Example 44, wherein the intermediate partial coil is a central intermediate partial coil, and wherein the plurality of partial coils further includes: an upper intermediate partial coil positioned between the upper partial coil and the central intermediate partial coil; and a lower intermediate partial coil positioned between the lower partial coil and the central intermediate partial coil.

In another example (“Example 47”), the brace system of Example 46, wherein the intermediate width is a central intermediate width, and wherein the upper intermediate partial coil has an upper intermediate width greater than the upper width and less than the central intermediate width.

In a further example (“Example 48”), the brace system of Example 46, wherein the intermediate width is a central intermediate width, and wherein the lower intermediate partial coil has a lower intermediate width less than the central intermediate width.

In another example (“Example 49”), the brace system of Example 44, wherein the brace system is a knee brace system, and the limb of the wearer is a leg of the wearer.

In one example (“Example 50”), a brace system is configured to be worn on a limb of a wearer. The system includes: a support member configured to receive the limb of the wearer; a spring hinge member coupled to the support member and having a medial side, a lateral side, and a width direction extending therebetween, the spring hinge member including: an upper base coupled to the support member; a lower base coupled to the support member; a plurality of partial coils coupling the upper base and the lower base, the plurality of partial coils including: an upper partial coil; a lower partial coil having lower width; and an intermediate partial coil disposed between the upper partial coil and the lower partial coil, the intermediate partial coil having an intermediate width, and the intermediate width being greater than the lower width.

In another example (“Example 51”), the brace system of Example 50, wherein the intermediate partial coil is a central intermediate partial coil, and wherein the plurality of partial coils further includes: an upper intermediate partial coil positioned between the upper partial coil and the central intermediate partial coil; and a lower intermediate partial coil positioned between the lower partial coil and the central intermediate partial coil.

In a further example (“Example 52”), the brace system of Example 51, wherein the intermediate width is a central intermediate width, and wherein the upper intermediate partial coil has an upper intermediate width less than the central intermediate width.

In another example (“Example 53”), the brace system of Example 51, wherein the intermediate width is a central intermediate width, and wherein the lower intermediate partial coil has a lower intermediate width greater than the lower width and less than the central intermediate width.

In a further example (“Example 54”), the brace system of Example 50, wherein the brace system is a knee brace system, and the limb of the wearer is a leg of the wearer.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a right side view of a brace system to be worn on the limb of a wearer, according to an embodiment of the present disclosure.

FIG. 2 is a left side view of the brace system of FIG. 1.

FIG. 3 is a perspective view of a hinge assembly of the brace system of FIG. 1.

FIG. 4 is a side view of the hinge assembly of FIG. 3.

FIG. 5 is a detail perspective view of the hinge assembly of FIG. 3.

FIG. 6 is a perspective view of a spring hinge member of the hinge assembly of FIG. 3.

FIG. 7 is a side view of the spring hinge member of FIG. 6.

FIG. 8 is a detail perspective view of the spring hinge member within line 8-8 of FIG. 6.

FIG. 9 is a detail perspective view of a partial chock of the spring hinge member of FIG. 6.

FIG. 10 is a detail perspective view of an alternative partial chock for the spring hinge member of FIG. 6.

FIG. 11 is a perspective view of a tension member of the hinge assembly of FIG. 3.

FIG. 12 is a perspective view of another tension member for the hinge assembly of FIG. 3, according to an embodiment of the present disclosure.

FIG. 13 is a perspective view of a spring hinge member of a hinge assembly for a brace system, according to another embodiment of the present disclosure.

FIG. 14 is a side view of the spring hinge member of FIG. 13.

FIG. 15 is a detail perspective view of the spring hinge member within line 15-15 of FIG. 13.

FIG. 16 is a perspective view of a spring hinge member of a hinge assembly for a brace system, according to another embodiment of the present disclosure.

FIG. 17 is a front view of the spring hinge member of FIG. 16.

FIG. 18 is a front view of an alternative spring hinge member of a hinge assembly for a brace system, according to another embodiment of the present disclosure.

FIG. 19 is a perspective view of a spring hinge member of a hinge assembly for a brace system, according to another embodiment of the present disclosure.

FIG. 20 is a detail perspective view of the spring hinge member within line 20-20 of FIG. 19.

FIG. 21 is a front view of the spring hinge member of FIG. 19 in a straight configuration.

FIG. 22 is a detail front view of the spring hinge member within line 22-22 of FIG. 21.

FIG. 23 is a front view of the spring hinge member of FIG. 19 in an angled configuration.

FIG. 24 is a detail front view of the spring hinge member within line 24-24 of FIG. 23.

FIG. 25 is an exploded perspective view of a spring hinge member of a hinge assembly for a brace system, according to another embodiment of the present disclosure.

FIG. 26 is a detail perspective view of the spring hinge member within line 26-26 of FIG. 25.

FIG. 27 is an exploded perspective view of a spring hinge member of a hinge assembly for a brace system, according to another embodiment of the present disclosure.

FIG. 28 is a detail perspective view of the spring hinge member within line 28-28 of FIG. 27.

FIG. 29 is a perspective view of a spring hinge member of a hinge assembly for a brace system, according to another embodiment of the present disclosure.

FIG. 30 is a detail perspective view of the spring hinge member within line 30-30 of FIG. 29.

FIG. 31 is a perspective view of a spring hinge member of a hinge assembly for a brace system, according to another embodiment of the present disclosure.

FIG. 32 is a detail perspective view of the spring hinge member within line 32-32 of FIG. 31.

### DETAILED DESCRIPTION

Various aspects of the present disclosure relate to brace systems that may be worn to protect and/or support a wearer's limb, including a joint, for example a wearer's knee. In some embodiments, knee brace systems may be used by a wearer who has suffered a meniscus tear, a patella injury, or has an unstable ligament, or an injury affecting lateral or torsional stability. In certain embodiments, knee brace systems include weight bearing capabilities, and such capabilities are changeable to accommodate different or varying training requirements, rehabilitation requirements, and wearer preferences.

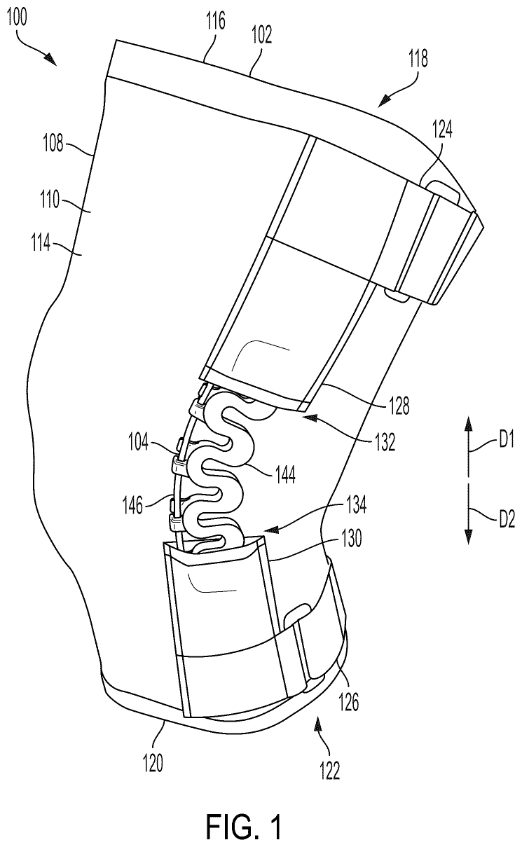

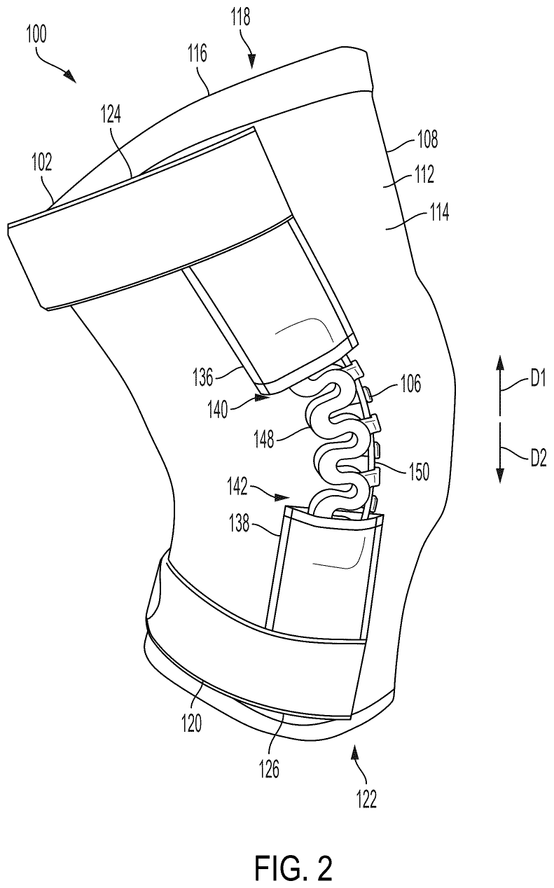

FIGS. 1 and 2 illustrate a brace system 100, according to an embodiment of the present disclosure. The brace system 100 generally includes a support member 102 that is configured to receive a limb of a wearer, a first or lateral hinge assembly 104 (FIG. 1) coupled to the support member 102, and a second or medial hinge assembly 106 (FIG. 2) coupled to the support member 102. The first hinge assembly 104 and the second hinge assembly 106 are biased toward relatively straight configurations, which causes the first hinge assembly 104 and the second hinge assembly 106 to apply forces to the support member 102 that urge the limb of the wearer in an extension direction. If worn on the leg of the wearer, for example, the first hinge assembly 104 and the second hinge assembly 106 thereby facilitate supporting the weight of the wearer.

Illustratively, the support member 102 includes a generally tubular or cylindrical sleeve 108 having a first side 110 (FIG. 1) and a second side 112 (FIG. 2), an inner surface (not shown), and an outer surface 114. The sleeve 108 also has a first or upper end 116 defining a first opening 118 and a second or lower end 120 defining a second opening 122. The inner diameter of the sleeve 108 may vary between the first end 116 and the second end 120. For example, the inner diameter of the sleeve 108 proximate the first end 116 may be sized to receive a portion of the upper leg of a user, such as a thigh. The inner diameter of the sleeve 108 proximate the second end 120 may be sized to receive the lower leg of a user, such as a calf or shin.

In various embodiments, the support member 102 may include one or more straps, illustratively a first or upper strap 124 and a second or lower strap 126, that facilitate fitting and securing the brace system 100 to the leg of the wearer. In various embodiments, the sleeve 108 may be constructed to be elastic and pliable and thereby fit snugly and provide compression to a user's leg when worn, and further to flex and move with the user's leg when the user runs, jumps or engages in other physical activity while maintaining the aforementioned snug fit at a suitable position on the leg. In various embodiments, the sleeve 108 may operate to wick sweat or other fluids away from a user's leg when worn. In various embodiments, the sleeve 108 may also be configured to have odor absorbing or odor prevention and/or anti-bacterial properties. In some embodiments, the sleeve 108 may have a suitable contour or shape to fit either a user's left leg or right leg. In some embodiments, the sleeve 108 may be a universal sleeve or interchangeable, that is, the sleeve 108 may be configured to be worn on either a user's left leg or right leg.

The sleeve 108 may be formed using any conventional or later-developed fabrication techniques. In various embodiments, for example, the sleeve 108 may comprise a woven or knit fabric, or may be constructed using any other technique suitable for forming flexible fabric materials.

The material(s) used to form the sleeve 108 can be chosen from any natural or synthetic classes of materials that provide appropriate flexibility, resiliency and manufacturability. In various embodiments, the sleeve 108 may be formed from synthetic elastic materials such as spandex, a polyester-polyurethane copolymer (commonly sold under the brand name Lycra®), or other comparable elastic material. In some embodiments, all or part of the sleeve 108 can include synthetic rubbers such as neoprene. In certain embodiments, a material that provides breathability, ventilation, and/or moisture wicking capability may be used. In some embodiments, the sleeve 108 may include a knit nylon or polyester material. In certain embodiments, the sleeve 108 may include a perforated material. In various embodiments, a plurality of different materials is used to construct the sleeve 108.

In some embodiments, the support member 102 may have different arrangements and/or constructions. For example, the support member 102 may include multiple sleeves, particularly a first or upper sleeve and a second or lower sleeve, that includes gaps therebetween and are only coupled by the hinge assemblies 104, 106. As another example, the sleeve 108 may include an aperture and/or buttress for receiving and/or supporting the patella of the wearer. As yet another example, the support member 102 may include an anterior or posterior opening that is selectively openable to receive the leg of the wearer and closeable around the leg of the wearer.

With further reference to FIGS. 1 and 2, the support member 102 includes one or more couplers for coupling to the first hinge assembly 104 and the second hinge assembly 106. In some embodiments and as illustrated, the couplers are pockets, more specifically four pockets that receive the first hinge assembly 104 and the second hinge assembly 106. The first side 110 (FIG. 1) of the support member 102 includes a first or upper lateral pocket 128 and a second or lower lateral pocket 130 that partially receive the first hinge assembly 104. The first lateral pocket 128 includes a first lateral opening 132 that generally faces in an inferior direction D1 and through which the first hinge assembly 104 extends. The second lateral pocket 130 includes a second lateral opening 134 that generally faces in a superior direction D2 and through which the first hinge assembly 104 extends. Similarly, the second side 112 (FIG. 2) of the support member 102 includes a first or upper medial pocket 136 and a second or lower medial pocket 138 that partially receive the second hinge assembly 106. The first medial pocket 136 includes a first medial opening 140 that generally faces in the inferior direction D1 and through which the second hinge assembly 106 extends. The second medial pocket 138 includes a second medial opening 142 that generally faces in the superior direction D2 and through which the second hinge assembly 106 extends.

With brief additional reference to FIGS. 1 and 2, the first hinge assembly 104 (FIG. 1) generally includes a first or lateral spring hinge member 144 and a first or lateral tension member 146, and the second hinge assembly 106 (FIG. 2) generally includes a second or medial spring hinge member 148 and a second or medial tension member 150. The first spring hinge member 144 and the second spring hinge member 148 carry bending loads upon flexion of the limb of the wearer, and the lateral tension member 146 and the medial tension member 150 carry tensile loads upon flexion of the limb of the wearer. In addition, the lateral tension member 146 and the medial tension member 150 are detachable and replaceable with tension members with different deformation resistance characteristics (provided by different dimensions (for example, unloaded length, cross section, or the like) or material properties (for example, elastic modulus)) to provide the system 100 with different load carrying characteristics (for example, depending on a wearer's training requirements, rehabilitation requirements, and/or preferences). These aspects and additional features of the first hinge assembly 104 and the second hinge assembly 106 are described below.

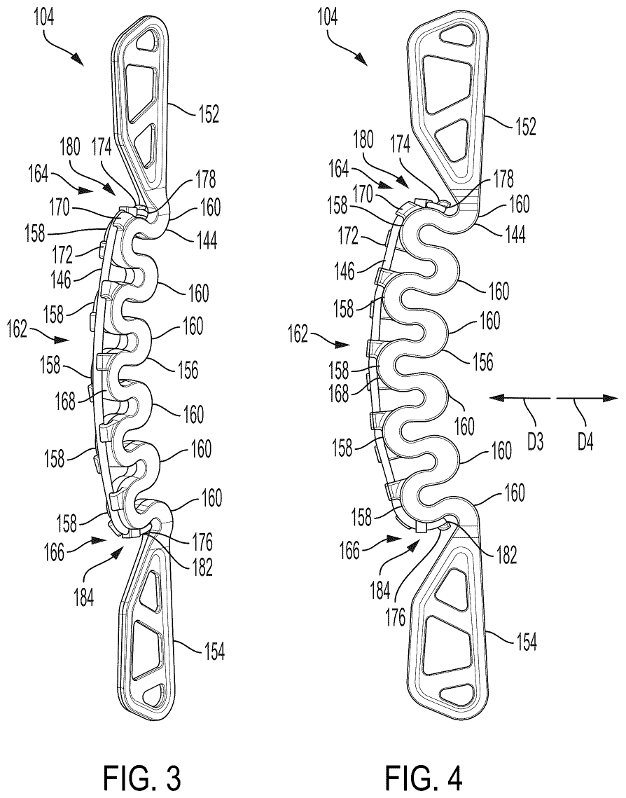

FIGS. 3 and 4 illustrate the first hinge assembly 104 separate from the support member 102 (shown elsewhere). Although the second hinge assembly 106 is not illustrated separate from the support member 102, it is understood that the first hinge assembly 104 may have the same or similar features to the first hinge assembly 104.

As described above, the first hinge assembly 104 includes the first spring hinge member 144 and the lateral tension member 146. The first spring hinge member 144 includes an upper base 152, a lower base 154, and an intermediate serpentine spring element 156 that couples the upper base 152 to the lower base 154. The first spring hinge member 144 may be constructed of various relatively stiff but resilient materials or composites, such as polymers. The first spring hinge member 144 may be a monolithic component, or the features of the spring hinge member 144, such as the upper base 152, the lower base 154, and the serpentine spring element 156, may be separate components that are joined together.

With continued reference to FIGS. 3 and 4, the upper base 152 and the lower base 154 are received in the upper lateral pocket 128 and the lower lateral pocket 130 (both shown elsewhere), respectively, of the support member 102. The upper base 152 and the lower base 154 illustratively include relatively flat, trapezoid-like shapes. However, the upper base 152 and the lower base 154 may alternatively have other relatively shapes that are generally inflexible in the plane of the spring hinge member 144 (that is, the plane of the page in FIG. 4).

The serpentine spring element 156 is generally flexible in the plane of the spring hinge member 144. To facilitate this flexibility, the serpentine spring element 156 includes a plurality of partial coils, and adjacent partial coils extend in opposite directions. Stated another way, the serpentine spring element 156 includes a plurality of anterior-facing partial coils 158, or coils with convex surfaces that generally face in an anterior direction D3 (FIG. 4). Each anterior-facing partial coil 158 is positioned between posterior-facing partial coils 160, or coils with convex surfaces that generally face in a posterior direction D4 (FIG. 4).

In some embodiments and as illustrated, the serpentine spring element 156, in an unloaded configuration, generally follows an arcuate path between the upper base 152 and the lower base 154, and an intermediate portion 162 of the serpentine spring element 156 is disposed anterior relative to an upper portion 164 adjacent the upper base 152 and a lower portion 166 adjacent the lower base 154. In other embodiments, the serpentine spring element 156 has different arrangements.

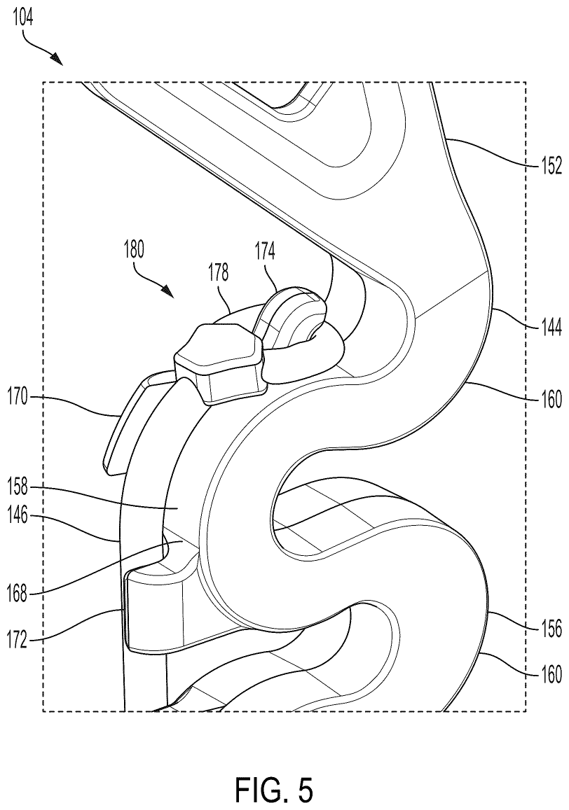

With continued reference to FIGS. 3 and 4 and additional reference to FIG. 5, the tension member 146 is positioned on an anterior side 168 of the serpentine spring element 156. The anterior side 168 of the serpentine spring element 156 includes a plurality of partial chocks 170, 172 (two of which are identified) that receive therebetween and position the tension member 146 along the serpentine spring element 156. The partial chocks 170, 172 are described in further detail below. With specific reference to FIGS. 4 and 5, the spring hinge member 144 further includes an upper bollard 174 adjacent the upper base 152 and a lower bollard 176 (FIG. 4) adjacent the lower base 154. The upper bollard 174 and the lower bollard 176 facilitate coupling the tension member 146 to the spring hinge member 144. More specifically, the upper bollard 174 receives an upper loop 178 of an upper end portion 180 of the tension member 146 and the lower bollard 176 receives a lower loop 182 of a lower end portion 184 (FIG. 4) of the tension member 146.

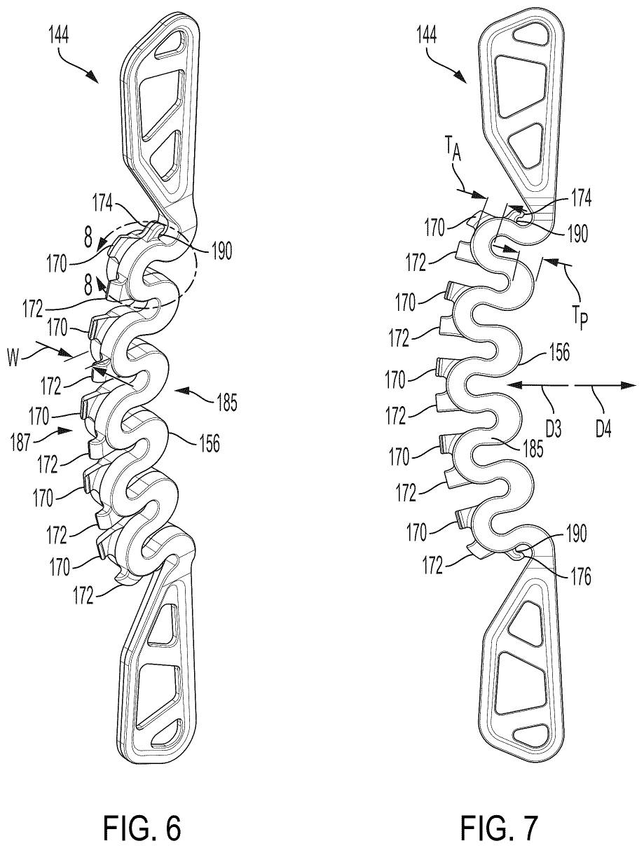

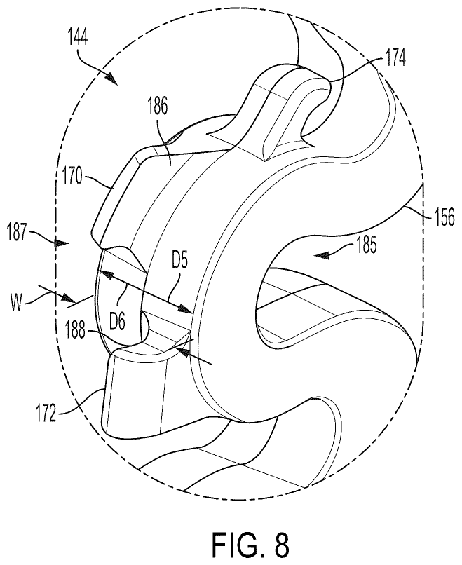

FIGS. 6-8 illustrate the first spring hinge member 144 separate from the tension member 146 and the support member 102 (both shown elsewhere). The serpentine spring element 156 has a width direction extending between a lateral side 185 and a medial side 187. In some embodiments and as illustrated, the serpentine spring element 156 has a uniform width W (FIGS. 6 and 8) proceeding from the upper base 152 and the lower base 154. In other embodiments and as shown elsewhere, the serpentine spring element 156 has a varying or non-uniform width W proceeding from the upper base 152 and the lower base 154. The serpentine spring element 156 also has a thickness direction that is perpendicular to the width direction. Referring specifically to FIG. 7, the anterior-facing partial coils 158 have a first thickness T_{A}, the posterior-facing partial coils 160 have a second thickness T_{P}, and the second thickness T_{P} is greater than the first thickness T_{A}.

FIGS. 6-8 also further illustrate the partial chocks 170, 172 and bollards 174, 176 coupled to the serpentine spring element 156. The partial chocks 170, 172 and bollards 174, 176 have shapes that facilitate coupling the tension member 146 to the serpentine spring element 156. For example and referring specifically to FIG. 8, each first partial chock 170 has a first concave surface 186 that contacts the tension member 146 and faces in a first direction (for example, a lateral direction D5), and each second partial chock 172 has a second concave surface 188 that contacts the tension member 146 and faces in a second, opposite direction (for example, a medial direction D6). Similarly and referring specifically to FIGS. 6 and 7, the upper bollard 174 and the lower bollard 176 each include a concave surface 190 that contacts the tension member 146 and faces in a third direction generally perpendicular to the first and second directions (for example, generally in the posterior direction D4).

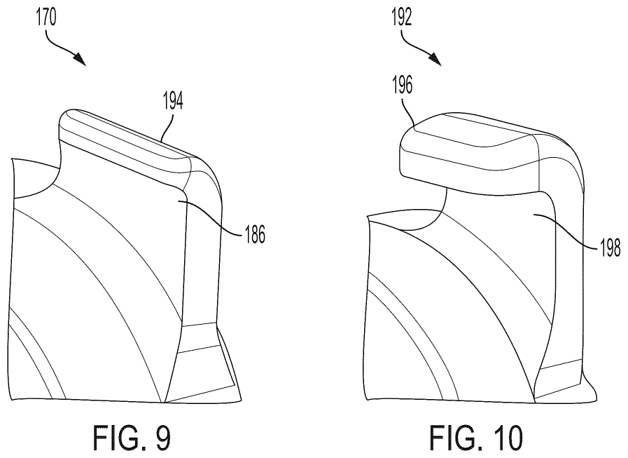

FIG. 9 further illustrates one of the first partial chocks 170 of the spring hinge member 144. In some embodiments, the partial chocks may be provided with different shapes and arrangements. For example, FIG. 10 illustrates another partial chock 192, which may be used in place of one or more of the first partial chocks 170 and/or second partial chocks 172. The partial chock 192 has a similar to shape to the first partial chock 170, except that the partial chock 170 has a relatively small awning portion 194 adjacent to its concave surface 186, and the partial chock 192 has a relatively large awning portion 196 adjacent to its concave surface 198. In certain embodiments, the relatively small awning portion 194 facilitates rapidly detaching the tension member 146 (shown elsewhere) from the spring hinge member 144, and the relatively large awning portion 196 facilitates further securing the tension member 146 to the spring hinge member 144.

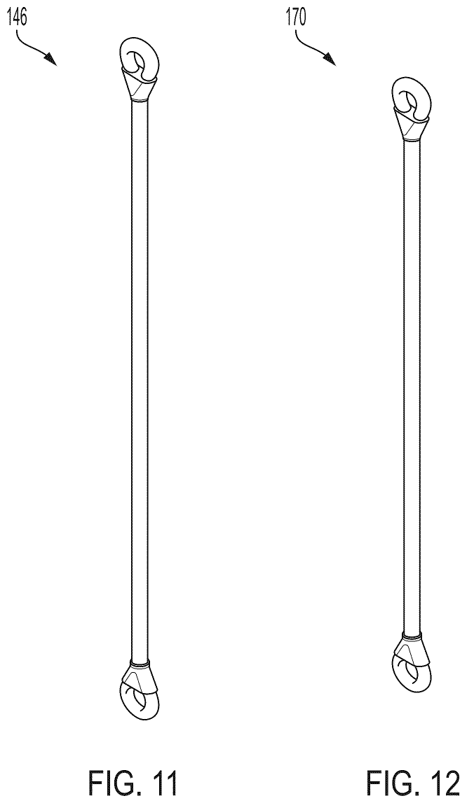

FIG. 11 illustrates the lateral tension member 146 separate from the other components of the hinge assembly 104. The tension member 146 is also referred to herein as a first tension member 146. As described above, the first tension member 146 is detachable from the system 100 and replaceable with other tension members having different deformation resistance characteristics (provided by different dimensions (for example, unloaded length, cross section, or the like) or material properties (for example, elastic modulus)) to provide the system 100 with different load carrying characteristics. FIG. 12 illustrates a second tension member 200 that is selectively attachable to and detachable from the hinge assembly 104. The second tension member 200 has a second resistance characteristic different than a first resistance characteristic of the first tension member 146, but is otherwise similar or substantially the same as the first tension member 146. In particular, the second tension member 200 has a shorter unloaded length than the first tension member 146.

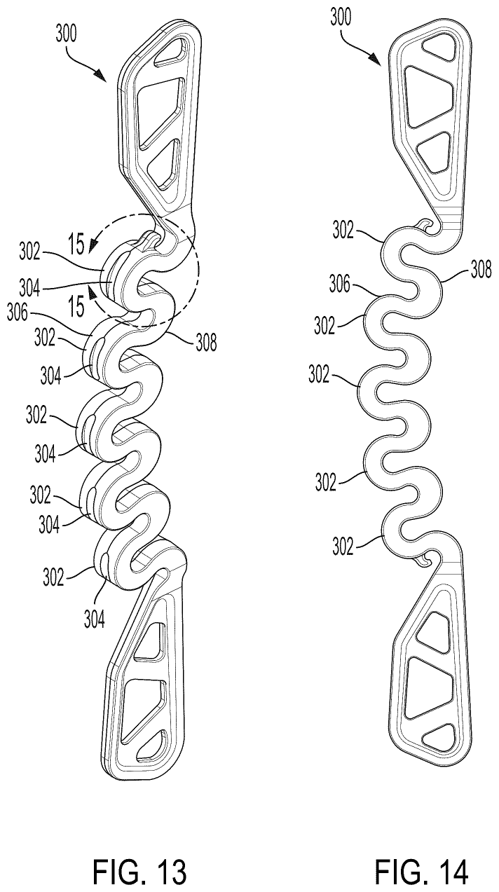

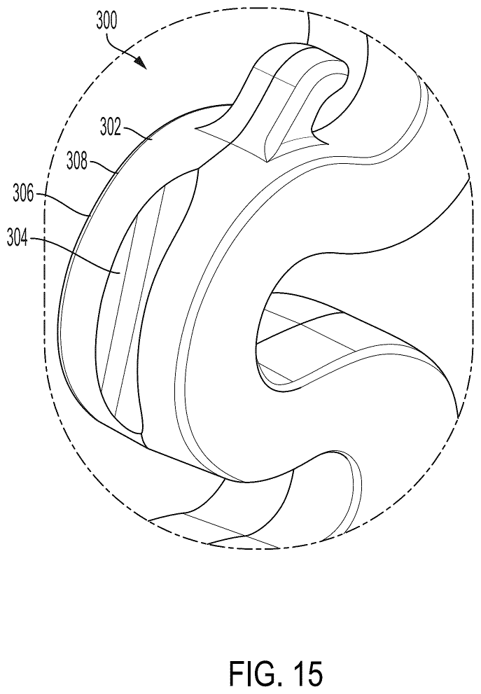

FIGS. 13-15 illustrate a spring hinge member 300, according to another embodiment of the present disclosure. The spring hinge member 300 may replace the spring hinge member 144 of the first hinge assembly 104 and/or the second hinge assembly 106 in the brace system 100. The spring hinge member 300 is substantially the same as the spring hinge member 144, except that the spring hinge member 300 lacks the partial chocks 170, 172, and the first partial coils 302 include generally superior/inferior extending grooves 304 (FIGS. 13 and 15) on the anterior side 306 of the serpentine spring element 308. The grooves 304 receive and position the tension member 146 along the serpentine spring element 308.

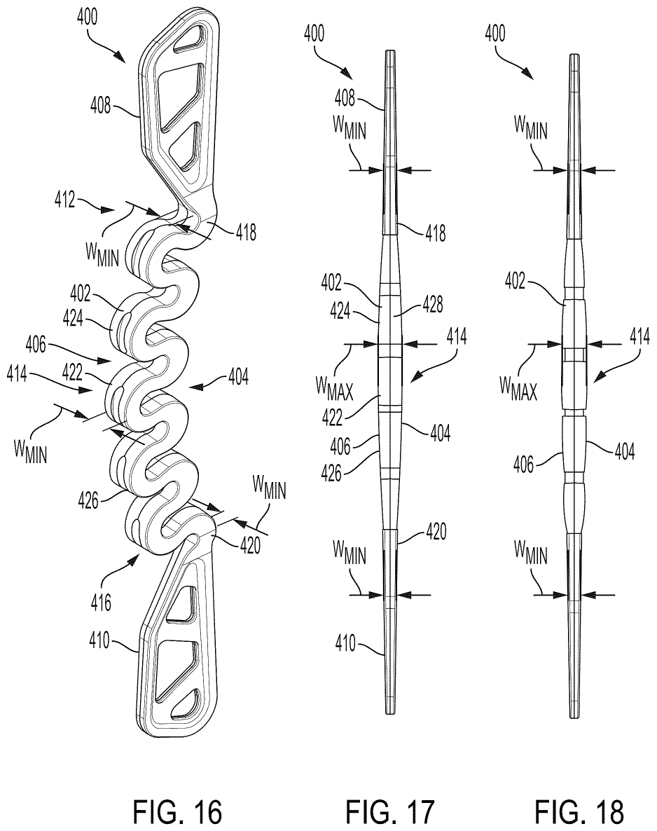

FIGS. 16 and 17 illustrate a spring hinge member 400, according to another embodiment of the present disclosure. The spring hinge member 400 may replace the spring hinge member 144 of the first hinge assembly 104 and/or the second hinge assembly 106 in the brace system 100. The spring hinge member 400 is substantially the same as the spring hinge member 144, except that the serpentine spring element 402 has a varying or non-uniform width, between the lateral side 404 and the medial side 406, proceeding from the upper base 408 and the lower base 410. For example and as illustrated, the serpentine spring element 402 may be relative narrow near the upper portion 412, relatively wide near the intermediate portion 414, and relatively narrow near the lower portion 416. Stated another way, the serpentine spring element 402 includes an upper partial coil 418 that includes an upper width and is coupled to the upper base 408, a lower partial coil 420 that includes a lower width and is coupled to the lower base 410, and an intermediate partial coil 422 that includes an intermediate width and is disposed between the upper partial coil 418 and the lower partial coil 420. The intermediate width is greater than the upper width and/or the lower width. Additionally, the intermediate partial coil 422 may be referred to as a central intermediate partial coil 422 and the intermediate width may be referred to as a central intermediate width. The serpentine spring element 402 may also include an upper intermediate partial coil 424 having an upper intermediate width and positioned between the upper partial coil 418 and the central intermediate partial coil 422, and a lower intermediate partial coil 426 having a lower intermediate width and positioned between the lower partial coil 420 and the central intermediate partial coil 422. The upper intermediate width may be greater than the upper width and less than the central intermediate width, and lower intermediate width may be greater than the lower width and less than the central intermediate width.

In certain embodiments and as illustrated, the intermediate partial coil 422 has the maximum width W_{MAX} of the serpentine spring element 402. Relatedly, the upper partial coil 418 and/or the lower partial coil 420 may have the minimum width W_{MIN} of the serpentine spring element 402; that is, the upper partial coil 418 and the lower partial coil 420 may have a common minimum width W_{MIN}.

Referring specifically to FIG. 17, due to the width variation of the serpentine spring element 402, the medial side 406 of the serpentine spring element 402 follows a curved path when viewed from the anterior side 428 of the spring hinge member 400, and the lateral side 404 of the serpentine spring element 402 follows a curved path when viewed from the anterior side 428 of the spring hinge member 400. The width may vary gradually proceeding toward the intermediate portion 414, as shown in FIG. 17, or alternatively, as shown in FIG. 18, the width may vary more abruptly toward the intermediate portion 414.

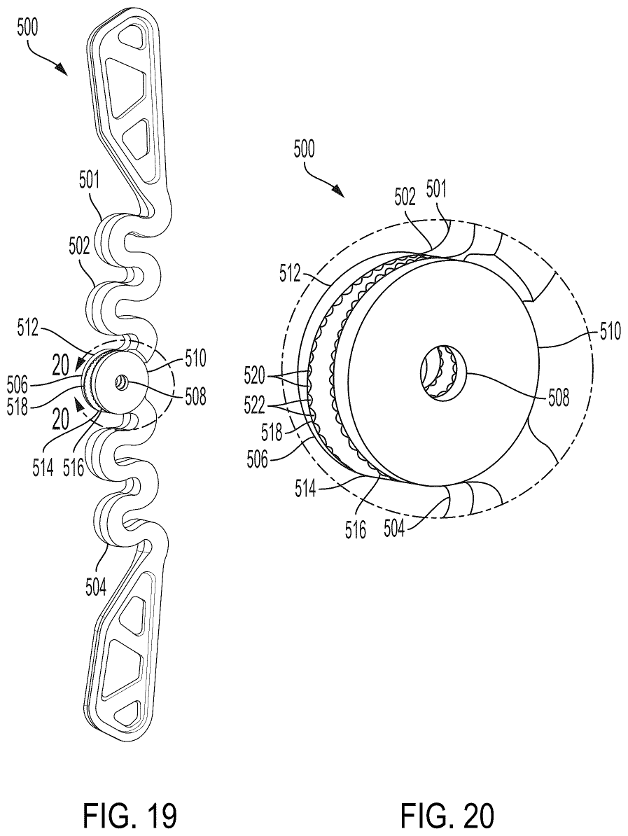

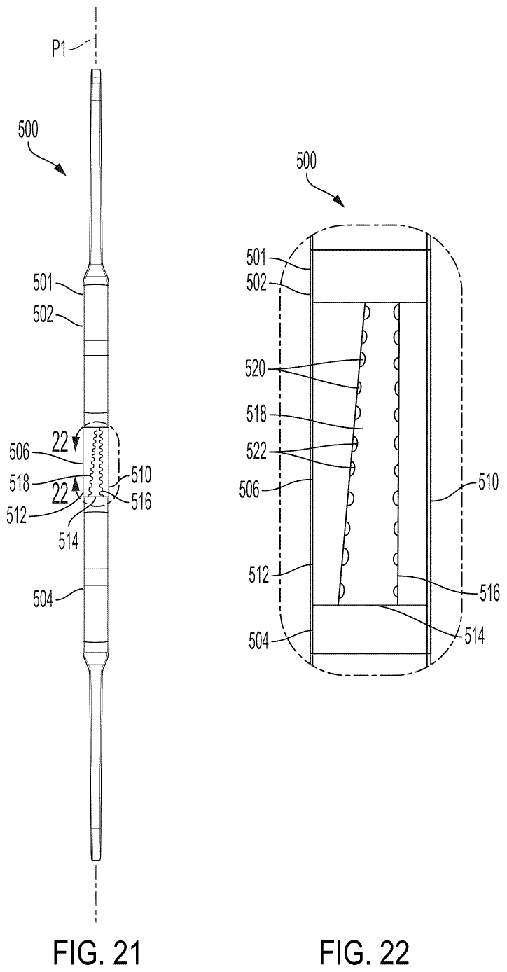

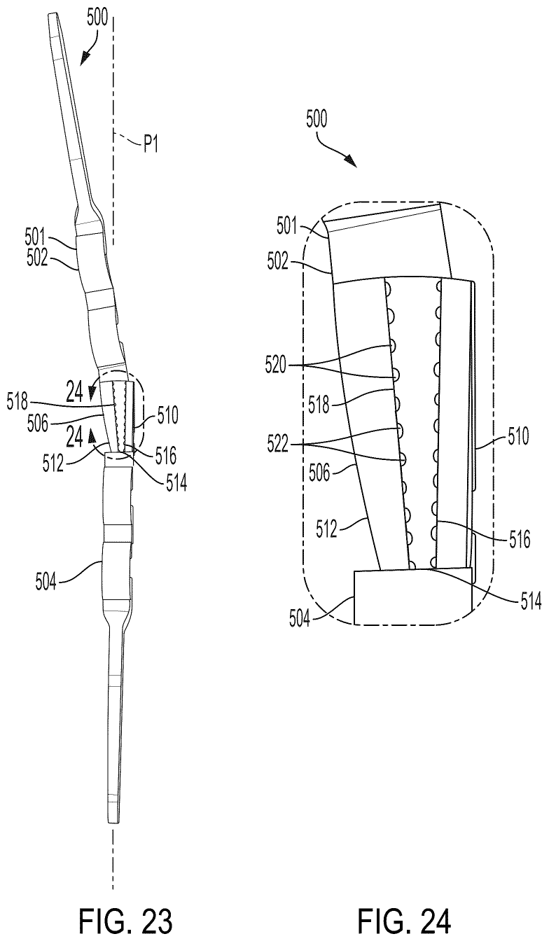

FIGS. 19-24 illustrate a spring hinge member 500, according to another embodiment of the present disclosure. The spring hinge member 500 may replace the spring hinge member 144 of the first hinge assembly 104 and/or the second hinge assembly 106 in the brace system 100. The spring hinge member 500 is substantially the same as the spring hinge member 144, except that the serpentine spring element 501 is divided into an upper spring portion 604 and a lower spring portion 504 that are coupled at an intermediate joint 506. A fastener (not shown) received in an aperture 508 of the joint 506 may normally secure the upper spring portion 604 and the lower spring portion 504. The joint 506 permits the wearer to selectively configure the spring hinge member 500, for example, by temporarily loosening or detaching the fastener, in a straight configuration (FIGS. 21 and 22), or a configuration in which both the upper spring portion 604 and the lower spring portion 504 are disposed in a common plane P1 (FIGS. 21 and 23—for example, a plane generally parallel to the sagittal plane of the wearer), or an angled configuration (FIGS. 23 and 24), or a configuration in which the upper spring portion 604 and the lower spring portion 504 are not disposed in the common plane P1. To facilitate such reconfiguration, one of the spring portions (for example, the lower spring portion 504) includes an upright disk 510, the other of the spring portions (for example, the upper spring portion 604) includes an angled disk 512, and a spacer 514 with a corresponding upright surface 516 and angled surface 518 is positioned between the disks 510, 512. The spacer 514 may be pivoted relative to the upright disk 510 and the angled disk 512 to reconfigure the spring hinge member 500 from the straight configuration to the angled configuration. Additionally and with specific reference to FIGS. 20, 22, and 24, the disks 510, 512 may include protrusions 520 and the spacer 514 may include recesses 522 for receiving the protrusions 520, or vice versa, to facilitate coupling the upper spring portion 604 and the lower spring portion 504 at specific orientations of the spacer 514, thereby permitting the upper spring portion 604 and the lower spring portion 504 to be offset from each other at various discrete angled configurations.

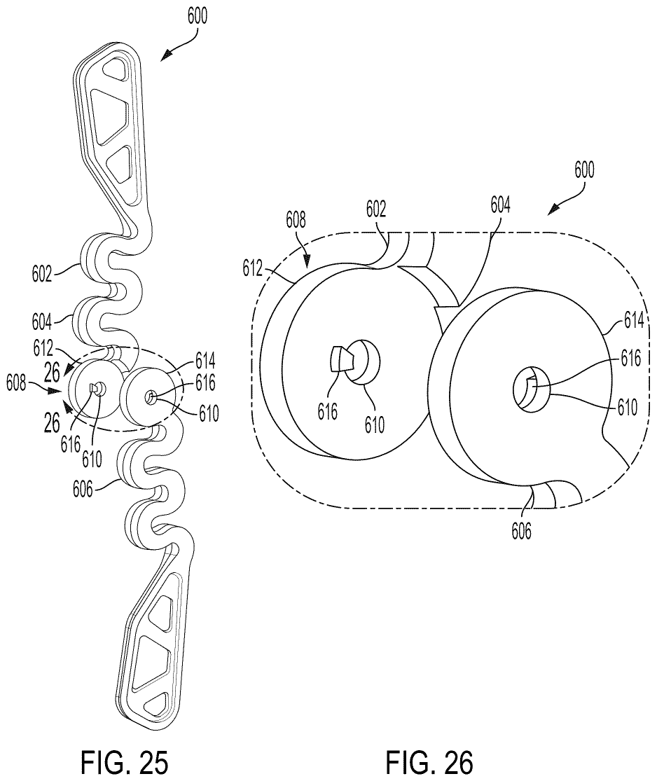

FIGS. 25 and 26 illustrate a spring hinge member 600, according to another embodiment of the present disclosure. The spring hinge member 600 may replace the spring hinge member 144 of the first hinge assembly 104 and/or the second hinge assembly 106 in the brace system 100. The spring hinge member 600 is substantially the same as the spring hinge member 144, except that the serpentine spring element 602 is divided into an upper spring portion 604 and a lower spring portion 606 that are coupled at an intermediate joint 608. A fastener (not shown) received in apertures 610 of the joint 608 may normally secure the upper spring portion 604 and the lower spring portion 606. The joint 608 permits the wearer to selectively adjust the angle between the upper spring portion 604 and the lower spring portion 606, for example, by temporarily loosening or detaching the fastener. Such adjustment alters the shape of the serpentine spring element 602 in its unloaded configuration, thereby adjusting the weight bearing characteristics of the spring hinge member 600. Illustratively, the joint 608 includes an upper disk 612 coupled to the upper spring portion 604 and a lower disk 614 coupled to the lower spring portion 606, and the disks 612, 614 are pivotable relative to each other. In certain embodiments and as illustrated, the disks 612, 614 include stops 616 to limit the angular adjustment range of the spring hinge member 600.

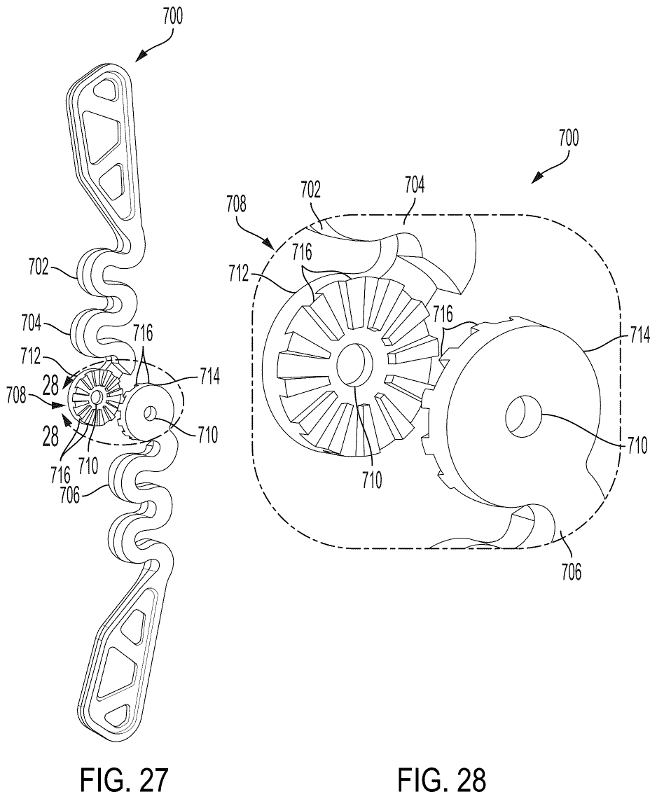

FIGS. 27 and 28 illustrate a spring hinge member 700, according to another embodiment of the present disclosure. The spring hinge member 700 may replace the spring hinge member 144 of the first hinge assembly 104 and/or the second hinge assembly 106 in the brace system 100. The spring hinge member 700 is substantially the same as the spring hinge member 144, except that the serpentine spring element 702 is divided into an upper spring portion 704 and a lower spring portion 706 that are coupled at an intermediate joint 708. A fastener (not shown) received in apertures 710 of the joint 708 may normally secure the upper spring portion 704 and the lower spring portion 706. The joint 708 permits the wearer to selectively adjust the angle between the upper spring portion 704 and the lower spring portion 706, for example, by temporarily loosening or detaching the fastener. Such adjustment alters the shape of the serpentine spring element 702 in its unloaded configuration, thereby adjusting the weight bearing characteristics of the spring hinge member 700. Illustratively, the joint 708 includes an upper disk 712 coupled to the upper spring portion 704 and a lower disk 714 coupled to the lower spring portion 706, and the disks 712, 714 are pivotable relative to each other. In certain embodiments and as illustrated, the disks 712, 714 include interdigitating protrusions 716 that permit the upper spring portion 704 and the lower spring portion 706 to be offset from each other at various discrete angled configurations.

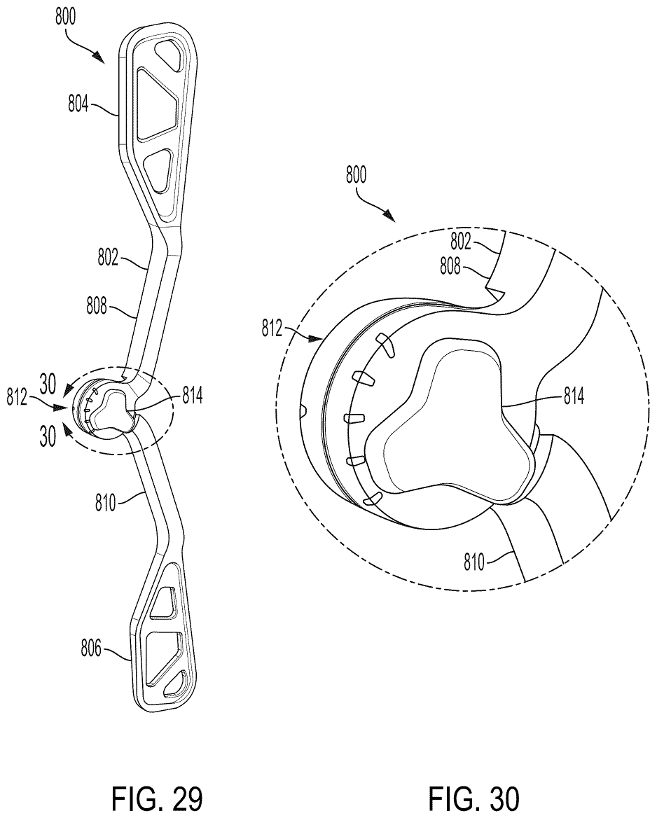

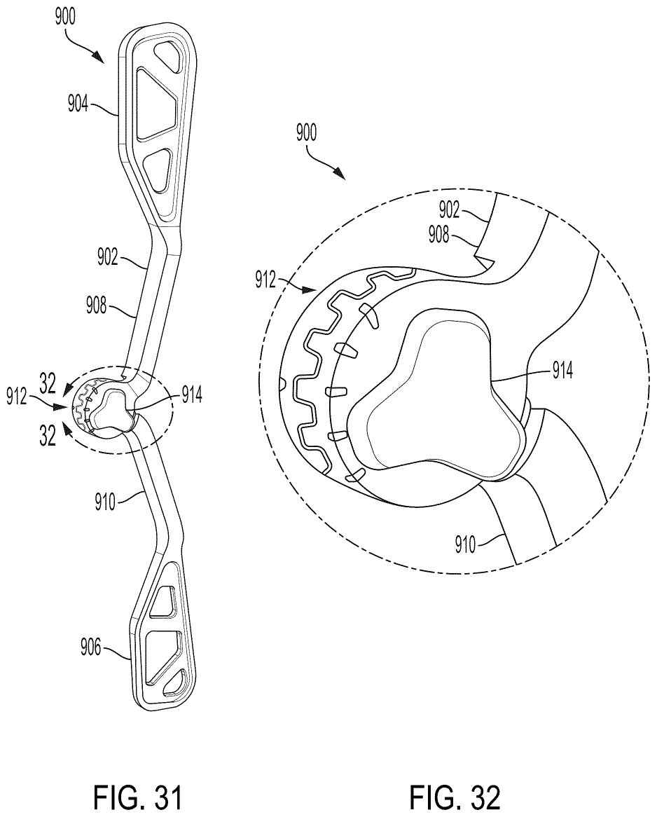

FIGS. 29 and 30 illustrate a spring hinge member 800, according to another embodiment of the present disclosure. The spring hinge member 800 may replace the spring hinge member 144 of the first hinge assembly 104 and/or the second hinge assembly 106 in the brace system 100. The spring hinge member 800 is substantially the same as the spring hinge member 144, except that an intermediate spring element 802 between the upper base 804 and the lower base 806 is divided into an upper spring portion 808 and a lower spring portion 810, which are coupled at an intermediate joint 812. The joint 812 includes a knob 814 that is pivotable to adjust the resistance to pivoting the upper spring portion 808 relative to the lower spring portion 810. Such adjustment alters the weight bearing characteristics of the spring hinge member 800.

FIGS. 29 and 30 illustrate a spring hinge member 900, according to another embodiment of the present disclosure. The spring hinge member 900 may replace the spring hinge member 144 of the first hinge assembly 104 and/or the second hinge assembly 106 in the brace system 100. The spring hinge member 900 is substantially the same as the spring hinge member 144, except that an intermediate spring element 902 between the upper base 904 and the lower base 906 is divided into an upper spring portion 908 and a lower spring portion 910, which are coupled at an intermediate joint 912. The joint 912 includes a knob 914 that is pivotable to adjust the resistance to pivoting the upper spring portion 908 relative to the lower spring portion 910. Such adjustment alters the weight bearing characteristics of the spring hinge member 900.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

What is claimed is:

1. A brace system configured to be worn on a limb of a wearer, comprising:
a support member configured to receive the limb of the wearer;
a hinge assembly coupled to the support member, the hinge assembly comprising:
a spring hinge member; and
a tension member;
wherein the spring hinge member and the tension member apply forces to the support member that urge the limb of the wearer in an extension direction.

2. The brace system of claim 1, wherein the spring hinge member comprises a serpentine spring element.

3. The brace system of claim 2, wherein the spring hinge member further comprises an upper base coupled to the support member and a lower base coupled to the support member, the serpentine spring element coupling the upper base to the lower base.

4. The brace system of claim 2, wherein the tension member is positioned on an anterior side of the serpentine spring element.

5. The brace system of claim 2, wherein the spring hinge member further comprises a plurality of partial chocks coupled to the serpentine spring element, the plurality of partial chocks receiving and positioning the tension member along the serpentine spring element.

6. The brace system of claim 1, wherein the spring hinge member includes a plurality of partial coils, wherein adjacent partial coils extend in opposite directions.

7. The brace system of claim 1, wherein the spring hinge member further includes an upper bollard receiving an upper end portion of the tension member and a lower bollard receiving a lower end portion of the tension member.

8. The brace system of claim 1, wherein the tension member is selectively attachable to and detachable from the spring hinge member.

9. The brace system of claim 1, wherein the tension member is a first tension member that is selectively attachable to the spring hinge member and the first tension member has a first deformation resistance characteristic when attached to the spring hinge member, and further comprising a second tension member that is selectively attachable to the spring hinge member and the second tension member has a second deformation resistance characteristic when attached to the spring hinge member, the second deformation resistance characteristic being different than the first deformation resistance characteristic.

10. The brace system of claim 1, wherein the brace system is a knee brace system, and the limb of the wearer is a leg of the wearer.

11. A brace system configured to be worn on a limb of a wearer, comprising:
a support member configured to receive the limb of the wearer;
a hinge assembly coupled to the support member, the hinge assembly comprising:
a spring hinge member configured to carry a bending load upon flexion of the limb of the wearer; and
a tension member configured to carry a tensile load upon flexion of the limb of the wearer.

12. The brace system of claim 11, wherein the spring hinge member comprises a serpentine spring element.

13. The brace system of claim 11, wherein the spring hinge member includes a plurality of partial coils, wherein adjacent partial coils extend in opposite directions.

14. The brace system of claim 11, wherein the spring hinge member further includes an upper bollard receiving an upper end portion of the tension member and a lower bollard receiving a lower end portion of the tension member.

15. The brace system of claim 14, wherein the upper end portion of the tension member includes an upper loop received by the upper bollard and the lower end portion of the tension member includes a lower loop received by the lower bollard.

16. The brace system of claim 11, wherein the tension member is selectively attachable to and detachable from the spring hinge member.

17. The brace system of claim 11, wherein the tension member is a first tension member that is selectively attachable to the spring hinge member and the first tension member has a first deformation resistance characteristic when attached to the spring hinge member, and further comprising a second tension member that is selectively attachable to the spring hinge member and the second tension member has a second deformation resistance characteristic when attached to the spring hinge member, the second deformation resistance characteristic being different than the first deformation resistance characteristic.

18. The brace system of claim 11, wherein the brace system is a knee brace system, and the limb of the wearer is a leg of the wearer.

19. A method for reconfiguring a brace system, the brace system comprising a support member, a spring hinge member coupled to the support member, a first tension member having a first deformation resistance characteristic when attached to the spring hinge member, and a second tension member having a second deformation resistance characteristic when attached to the spring hinge member, the second deformation resistance characteristic being different than the first deformation resistance characteristic, the method comprising:
detaching the first tension member from the spring hinge member; and
thereafter attaching the second tension member to the spring hinge member.

20. The method of claim 19, wherein the spring hinge member includes a serpentine spring element, and attaching the second tension member to the spring hinge member includes positioning the second tension member along the serpentine spring element.
